(19) United States
(12) Patent Application Publication
(10) Pub. No.: US 2002/0077691 A1
(43) Pub. Date: Jun. 20, 2002

(54) Ostial stent and method for deploying same

(75) Inventor: John C. Nachtigall, Sunnyvale, CA (US)

Correspondence Address: FULWIDER PATTON LEE & UTECHT, LLP, Howard Hughes Center, Tenth Floor, 6060 Center Drive, Los Angeles, CA, 90045, US

(73) Assignee: ADVANCED CARDIOVASCULAR SYSTEMS, INC.

(21) Appl. No.: 09/740,116

(22) Filed: Dec. 18, 2000

(51) Int. Cl. A61F 2/06

(52) U.S. Cl. 623/1.12; 623/1.19; 623/1.36

(57) ABSTRACT

An expandable stent to be positioned and implanted at the intersection of a great vessel and a branch vessel. A deployable stop engages the wall of the great vessel surrounding the ostium and the stent body extends into and is implanted in a branch vessel. A sheath holds the stent in a compressed state during delivery and a retainer holds the stop in an undeployed position while the delivery system is advanced to a desired location at the intersection of the great vessel and the branch vessel.

### BACKGROUND OF THE INVENTION

The present invention relates to intravascular stents and methods for their deployment, and more specifically, to a stent for treating a diseased branch vessel adjacent to another great vessel, such as the aorta.

The American Heart Association estimates that cardiovascular disease takes the lives of almost 960,000 Americans each year. (Phil Davis, Affairs of the Heart, Los Angeles Daily News, Sep. 6, 1999). Diseases of the vascular system may occur as a result of several etiologies that lead to the development of atherosclerosis, a disease of the arteries characterized by thickening, loss of elasticity, and calcification of arterial walls, which manifests itself in two predominant forms. In one form, a narrowing of blood vessels impedes blood flow through the vessel lumen. In another form, arterial walls degenerate due to the formation of aneurisms, which cause the walls of the affected artery to weaken and balloon outward by thinning. Many patients with vascular disease choose to explore treatments that do not require surgery, such as cholesterol reducing regimens or drugs, beta blockers to regulate and reduce blood pressure, and blood-thinning agents. In the United States, more than 850,000 angioplasties and bypasses are performed annually at a cost of around $30 billion. (Paul Engstrom, Broken Hearts, The Wall Street Journal, Jun. 5, 2000).

Angioplasty is a less invasive alternative to bypass surgery and is a procedure where a balloon-tipped catheter or other device is used to enlarge a narrowing in a artery. This enlargement is accomplished by radially compressing the atherosclerotic plaque of a stenosis against the inside of the artery wall, which dilates the lumen of the artery. The most common angioplasty procedure is Percutaneous Translumenal Coronary Angioplasty (PTCA), a well-known procedure in which an occluded coronary artery is dilated by inserting a balloon catheter through the skin and through the lumen of the vessel to the site of the narrowing, where the balloon is then inflated to compress the plaque and restore normal blood flow through the artery.

Although angioplasty procedures are widely accepted for treatment of occluded arteries, the problem of restenosis following the angioplasty treatment is a complication a patient must face. Restenosis is the reclosure or renarrowing of an artery following trauma caused by surgical attempts to open an occluded portion of the artery, and is frequently caused by the elastic rebound of the arterial wall and/or by dissections in the vessel wall caused by the angioplasty procedure. To combat restenosis and maintain the patency of the vessel lumen, vascular surgeons implant tubular supports known as stents into surgically repaired vessels.

Stents are used to tack-up dissections in vessel walls and to prevent the elastic rebound of repaired vessels, thereby reducing the level of restenosis for many patients. The stent is typically inserted by catheter into a vascular lumen at an easily accessible location, such as the brachial or femoral arteries, and then is advanced through the vasculature to the deployment site. The stent is initially maintained in a radially compressed or collapsed state to enable it to be maneuvered through the body lumen, and is mounted to a delivery system for advancement through a patient's vasculature to the deployment site. Once the stent has reached the stenotic site within a damaged vessel, and is ready for deployment, it is expanded by internal means or by means integral to the delivery system that are well known in the prior art. In its expanded state, the stent provides internal support for the vessel lumen and reduces the likelihood of the development of restenosis.

Placement of the stent within the vasculature can be especially challenging when the stenotic region is near the intersection of two vessels. For example, the placement of a stent to repair a diseased vessel that is a branch vessel, such as the renal artery, near its ostium with a great vessel, such as the aorta, is particularly challenging because the stent must be securely positioned in an area that supports a heavy volume of blood flow without occluding the blood flow in either the branch vessel or the great vessel. Additionally, the angle created by the intersection of a great and a branch vessel can lead to difficulties in precisely positioning the stent in the damaged branch vessel. Vascular surgeons often have difficulty aligning the stent to optimally repair the stenotic region of such a branch vessel, which leads to placement of the stent within the branch vessel such that a portion of the stent extends into the great vessel. This can result in both occlusion of the blood flow through the great vessel and shifting of the stent. Finally, heavy blood flow into and through the branch vessel may directly lead to shifting of the stent after it has been positioned and expanded within the vessel lumen.

Conventional stents are designed to repair areas of blood vessels that are generally located somewhere along the length of single elongated vessel, and as such, they are not sufficiently equipped to be reliably and securely placed at a site that has a substantially perpendicular intersection and that supports a heavy volume of blood flow. Use of such conventional stents in the vicinity of vessel intersections may lead to undesirable shifting of the stent within the vessel.

Prior art stents have incorporated various arrangements to assist in securing the expanded stent to the walls of the vessel lumen in a stenotic region. Examples of such arrangements include rounded protrusions, longitudinal rails, or tines configured to project in some manner from the tubular body of the stent itself and grip into the walls of the vessel lumen. Prior art designs also have proposed attachments of securing components to the ends of the stent's body, as opposed to the body itself. For example, flaps have been used to assist in securing the stent within a conventional vessel's stenotic region, or a flaring portion has been used to cap the ostium of a diseased bifurcated vessel. The foregoing designs, however, while helpful in securing stents in conventional vessels, or even capping bifurcated vessels, do not adequately address the need for a reliable means of securing a stent in the ostium of a vessel branching off substantially perpendicular to a great vessel.

There has been no adequate response yet to the need for a stent that is reliably secured in the ostium of a diseased branch vessel such that the stent is able to endure the heavy blood flow to the vessel without further occluding flow through the branch vessel or the great vessel.

### SUMMARY OF THE INVENTION

The present invention provides a stent that is delivered through a great vessel to repair a branch vessel that is diseased in the vicinity of its ostium with the great vessel. By engaging the walls of the great vessel surrounding the ostium, the stent is reliably secured in the branch vessel without obstructing blood flow through either the great vessel or the branch vessel. The present invention also provides a method and delivery system for delivering and securely implanting the stent in the branch vessel.

The stent of the present invention is characterized by an elongated expandable tubular body with a deployable stop adjacent its proximal end for engaging the ostium created by the intersection of a great vessel, such as the aorta, and a branch vessel, such as the renal artery. The stent is delivered intralumenally through a great vessel, and the stop is deployed to abutt the wall of the great vessel around the ostium as the distal end of the tubular body extends into the branch vessel and beyond its diseased portion.

The tubular body of the stent is capable of radial expansion to increase its cross-sectional area and engage the walls of the branch artery. In one embodiment, the cross-sectional area of the stent is increased by its self-expanding material composition, but the stent may be expanded by exerting force upon the internal walls comprising the tubular body. It is contemplated that the tubular body is constructed of a material with sufficient radial strength to allow it to assume its reduced pre-expanded cross-sectional area and to, once expanded, also retain its expanded and implanted cross-section. This radial strength may be provided by a combination of the geometric structural configuration chosen and by the selection of material forming the tubular body. The material of the tubular body should have a high space-to-metal ratio.

The stop may be deployed radially outwardly from the proximal end of the stent by a deployment means that biases it to rotate radially outwardly. In one embodiment, the stop and the proximal end of the stent are formed of one piece, but it may be a separate element disposed adjacent such proximal end. In another embodiment, the stop is composed of the same material as the stent tubular body, but it may be composed of a material with greater radial strength to lend greater rigidity. In one embodiment, the stop comprises a plurality of elongated stop wings that are each capable of deployment from an undeployed position, with the wings resting against the exterior of the stent body or a central tubular member which mounts the stent, to a deployed position, projecting radially outwardly from such body or member. In this embodiment, the stent is constructed with thermally responsive hinges, which rotate the stop wings to the undeployed position at temperatures below normal body temperature, and are responsive to elevated temperatures corresponding with the body temperature to project the stop wings perpendicular to the longitudinal axis of the stent body. In an alternative embodiment, such wings are biased to the deployed position, and are held in their undeployed position by an external restraining device during delivery. Removal of the restraint then deploys the stop wings to their transversely projecting state. The stop is constructed to, in its deployed position, project substantially perpendicular to the longitudinal axis of the stent, and is of sufficient length to abutt the wall of the great vessel surrounding the ostium to limit entry of such stent into the branch vessel.

Placement of the stent within the branch vessel is accomplished by delivering the stent, in its radially compressed state with the stop in the undeployed position, intralumenally through the great vessel until the stent is positioned at the ostium to the branch vessel. The stop is then deployed, and the distal end of the stent advanced into the branch vessel until the stop engages the wall of great vessel surrounding ostium. The stent is then expanded within the branch vessel. As the stop engages the walls surrounding the ostium, it acts in consort with the expanded stent engaging the walls of the branch vessel to firmly secure the stent in its desired position.

Expansion and deployment of the stent can be accomplished through its own self-expanding material composition or through the utilization of a separate expander such as a catheter balloon. In one embodiment the tubular body of the stent is composed of temperature responsive material that expands the stent after a greater quantity of heat transfer than required to rotate the thermally responsive hinges to the deployed position. For example, the stent tubular body and the stop may be formed from shape-memory metal or a thermally responsive material, such as nickel-titanium (NiTi). At reduced temperature, the stent remains in an unexpanded state and the stop remains in its undeployed state substantially parallel to the longitudinal axis of the stent. When the stent is advanced into the aorta, it is heated to appropriate body temperature causing the thermally responsive hinge to rotate the stop to its laterally projecting deployed position. As the stent is then advanced into the branch vessel, the stop engages the wall surrounding the ostium to stop the stent body registered with the diseased portion of the branch vessel wall. As a patient's normal body temperature continues to expand the stent body to its expanded configuration, the stent body engages the walls of the diseased branch vessel. In an alternative embodiment, the stent tubular body and stop are formed from a superelastic or pseudoelastic alloy, such as NiTi, which forms stress induced martensite upon compression. When the compressive force is removed, the material converts to stable austenite. Thus, for example, the stent and stop are compressed into a sheath where stress induced martensite is formed and the metal is malleable and flexible for intralumenal delivery. When the sheath is removed, the stress is relieved and the metal converts to stable austenite so the stop opens and the stent expands.

It is also contemplated that the stent body may be composed of nickel-titanium, with the stop composed of a resilient material that is not memory-retaining. In this embodiment, the stop is radially outwardly biased, and is normally held in its undeployed position by an external restraining device. In alternative embodiments, when an external restraining device such as a sheath is employed, it is contemplated that the stent and the stop be composed of a spring like self-expanding material such that the stent is capable of self-expansion upon removal of the restraining device. A stent composed of material that is not self-expanding may also be incorporated when it is designed for use with the balloon of a balloon catheter and a stop that is held in its undeployed position by an external restraining device. When the restraining device is removed, the stop deploys to engage the great vessel walls surrounding the ostium. Then, when the stent has been positioned in the branch vessel as desired, the balloon is expanded to expand the stent within the vessel lumen and position the stent to cover the diseased portion of the vessel.

Alternatively, the stent body and the stop wings can be formed of a resilient material such as stainless steel that has been heavily cold worked so that the material acts as a spring. For example, the stainless steel can be cold worked so that the stent can be compressed and the stop wings compressed against a catheter shaft and held in place by a sheath that covers the stent and the stop wings. When the external restraining device such as a sheath is withdrawn after positioning the catheter in the target vessel, the stent will self expand and the stop wings will deploy to engage the great vessel wall surrounding the ostium.

The method of delivering the stent of the present invention involves accessing the patient's diseased branch vessel, such as the renal artery, intraluminally through a great vessel, such as the aorta. In the case of a self-expanding stent body and stop, both are held therein in a compressed state to be advanced in the aorta towards the ostium of the renal artery. The stop is then deployed to project perpendicularly so that, upon advancement of the stent body into the renal artery, such stop will engage the wall of the aorta surrounding the ostium to block such stent from further advancement. This then serves to register the stent body with the diseased renal artery wall at the root adjacent such ostium so that, upon release, it will engage such diseased wall.

Other objects and features of the invention will become apparent from consideration of the following descriptions, taken in conjunction with the accompanying drawings, which illustrate by way of example the features of the invention.

### BRIEF DESCRIPTION OF THE DRAWINGS

FIG. 1 is a side view of a stent embodying the present invention in the undeployed position being delivered through a great vessel to the ostium of a diseased branch vessel.

FIG. 2 is a side view, similar to FIG. 1, depicting the stop in its deployed position.

FIG. 3 is a side view, similar to FIG. 2, with the stop of the deployed stent body positioned within the branch vessel.

FIG. 4 is a side view, similar to FIG. 3, with the stent body expanded and the delivery system removed.

FIG. 5 is an end view, taken along line 5-5 of FIG. 4.

FIG. 6 is a longitudinal view, taken along the line 6-6 in FIG. 1, depicting the stent of the present invention with the stent body compressed and the stop retracted during delivery.

FIG. 7 is a transverse sectional view, taken along the line 7-7 of FIG. 6.

FIG. 8 is a transverse sectional view, taken along the line 8-8 of FIG. 6.

FIG. 9 is a longitudinal sectional view, taken along the line 9-9 of FIG. 2, depicting the stop in the deployed position and stent body retracted.

FIG. 10 is a longitudinal sectional view taken along the line 10-10 of FIG. 3, depicting the stent body in the process of being released.

FIG. 11 is a longitudinal sectional view similar to FIG. 10, depicting the stent body in its expanded and implanted state.

### DETAILED DESCRIPTION OF THE PREFERRED EMBODIMENTS

As shown in the drawings, the present invention is embodied in an ostial stent 15 that includes, generally, an elongated tubular body 40 having at its proximal extremity an elongated deployable stop 50 projecting therefrom to abut the wall of an aorta 20 to limit entry of such tubular body into a diseased branch vessel 22.

Typically, the stent includes a longitudinal tubular body 40 in the form of a cylindrical shell. The material and structure of the stent 15 is selected to ensure that the body is expandable radially outwardly with sufficient force to engage the vessel wall 23 and to retain both its pre-expanded and post-expanded shape and cross-sectional area. It is contemplated that this material have a high space-to-metal ratio, and also that the geometric design of the stent 15 facilitates its expansion and shape retention. In one embodiment, the stent body 40 is constructed of a thermally responsive material, and is sized to, upon deployment as shown in FIG. 4, assume the size and shape of the interior wall 23 of a branch vessel 22 branching off from a great artery 20, such as a renal artery branching off from an aorta.

With continued reference to FIG. 4, a deployable stop 50 projects from the proximal extremity of the stent 15, extending radially outwardly in a transverse relationship to the longitudinal axis of the stent tubular body 40. The stop 50 is formed as a single piece with the stent tubular body 40, or, in the alternative, may be a separate element attached adjacent to the tubular body proximal extremity. The stop is biased to rotate radially outwardly to this transversely projecting deployed position by a deployment means 54 comprising a thermally responsive hinge 42. In one embodiment, the stop is composed of the same self-expanding, thermally responsive material as the stent 15, which enhances its radially outwardly biased tendency when exposed to a patient's body temperature.

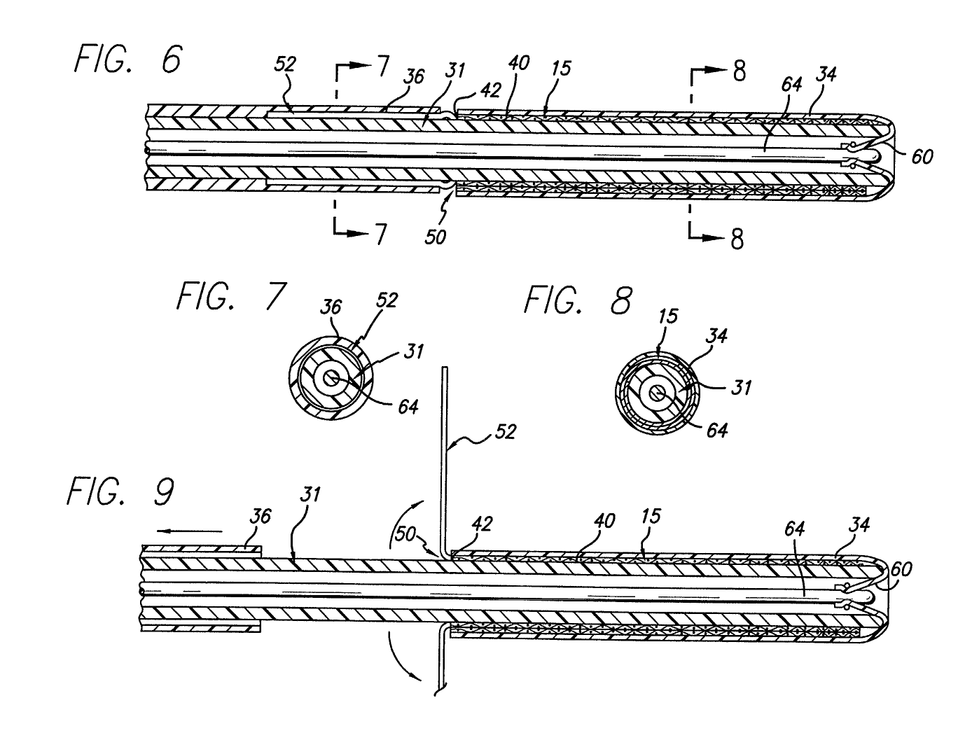

Referring now to FIG. 5, in one embodiment, the deployable stop 50 is formed by a plurality of radially outwardly projecting resilient stop wings 52 arrayed circumferentially equidistant about the proximal extent 42 of the cylindrical stent tubular body 40, and it is contemplated that three such stop wings 52 may provide the best structural integrity and simplicity for such an embodiment. Each individual stop wing 52 is connected to the proximal extent of the tubular body by means of a thermally responsive hinge 42. Such hinges are designed to retain the stop wings 52 in an undeployed position 56, as shown in FIG. 6, wherein the wings rest against the tubular body or a central tubular member 31 of the delivery system 28 such that the stop wings project longitudinally to the axis of the body when the hinges are maintained at below body temperature. When the hinges 42 are exposed to elevated temperatures corresponding with a patient's body temperature, the hinges rotate the stop wings to a deployed position 58 perpendicular to the axis of the stent body. Referring again to FIG. 4, when the stop wings are in this deployed position, they may engage the interior wall of the aorta 25 surrounding the ostium 24 of the renal artery 22 to block further insertion of the stent and to register the stent body 40 with the diseased area 26.

Referring to FIGS. 4 and 5, the stop wings 52 are generally in the form of elongated spokes, being generally rounded at their distal ends. It is contemplated that each stop wing may be of any suitable shape and dimension to best engage the wall of the great vessel 21 surrounding the ostium 24. It is also contemplated that the external edges of the stop wings that engage the great vessel wall 25 may be formed with teeth to more securely grip the great vessel wall. The stop wings may be shorter in length than the stent tubular body 40, but are of sufficient length to securely engage the great vessel wall 25 to positively block travel of such stent body into the branch vessel 22 beyond the diseased area 26 thereof. The stop wings are generally rigid, however, it is contemplated that they may be somewhat flexible or may have a shape memory to assume, in transverse section and in the undeployed position, a concave configuration complementing that of the exterior surface of the stent tubular body 40 or the central tubular member 31 of its delivery system 28.

As shown in FIG. 6, when the stent 15 and stop wings 52 are configured for delivery, the stop wings are bent radially inwardly from their outwardly biased transverse orientation and must remain in position substantially parallel to the longitudinal axis of the stent 15. Therefore, it is contemplated that the material comprising the thermally responsive hinges 42 joining the individual stop wings must be sufficiently malleable to withstand the radially inwardly directed force created by securing the stop wings in their undeployed position 56 at temperatures below body temperature, but sufficiently rigid and elastic to return the stop wings to the transversely projecting orientation of their deployed position 58 upon exposure to temperatures at or above normal body temperature.

It is contemplated that various deployment/delivery systems 28 familiar to those skilled in the art may be suitable for the deployment of the stent 15 of the present invention. As shown in FIGS. 1-3, such a system may include a delivery catheter (not shown) having a central tubular member 31 configured at its distal extremity with a stent mounting region 29 about which the stent 15 will be compressed, with the stop wings 52 in their undeployed position as shown in FIGS. 1 and 6, for delivery. Referring again to FIGS. 1-3, such a central tubular member 31 may receive telescopically over its proximal end a stop container tube 36 having a distal extremity configured to telescope distally over the stop wings when they are disposed in their undeployed position 56. As shown in FIG. 6, in one embodiment, the stop wings may be held by such stop container tube 36 in the undeployed position wherein they are rotated radially outwardly away from the stent body 40 until they rest longitudinally against the central tubular member 36. It is also contemplated, however, that the stop wings may be held in an undeployed position 56 wherein they are rotated radially inwardly to rest longitudinally against the tubular body 40 of the stent, as both the undeployed stop wings and compressed stent 15 are encompassed by an external restraining device, dispensing with the requirement for the stop container tube 36.

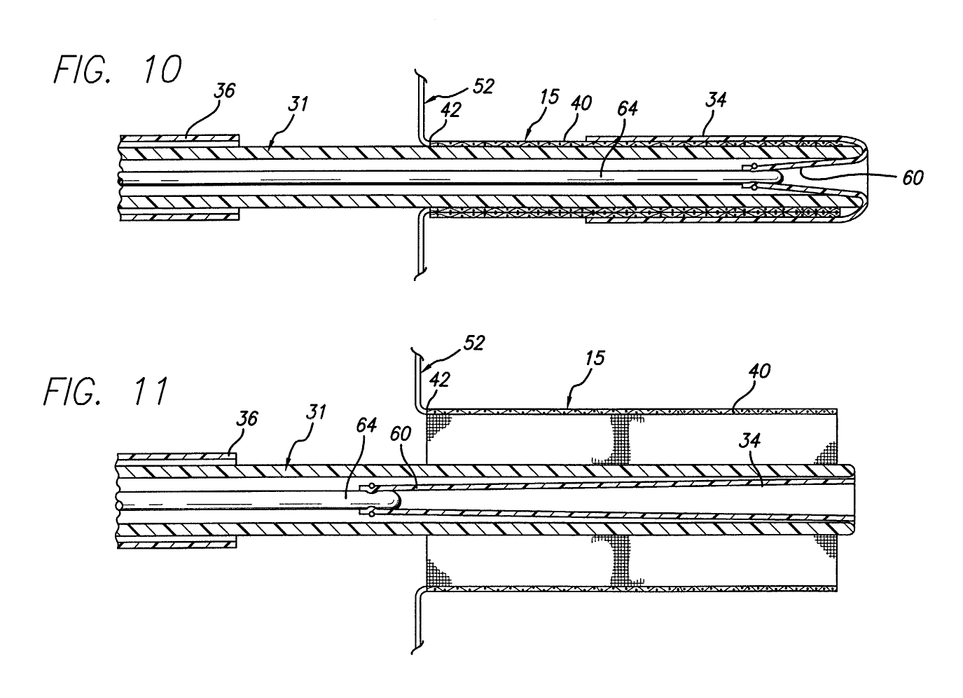

The delivery system 28 may further include a tubular sheath 34, as shown in FIGS. 6 and 9, configured to be complementally received over the exterior of the stent body 40 that encloses at its distal extremity radially inwardly projecting tethers 60. Such tethers are secured to the distal end of a retractor wire 64 that telescopes longitudinally through the internal diameter of the central tubular member 36. The sheath is constructed similar to those of conventional catheter systems used for angioplasty procedures, and may be made of suitable polymers, such as polyethylene, polyester, polymide and the like which are well known to those skilled in the art. The sheath is also flexible, and is configured to be disposed in close relationship with the retracted stent body 40 as shown in FIG. 6. As shown in FIG. 10, it is further constructed to, upon retraction of the retractor wire 64 proximally, be drawn distally over the distal end of the central tubular member 36, essentially turning the sheath inside out as it is withdrawn proximally into the interior of the central tubular member to the position shown in FIG. 11, thus releasing the stent body 40.

In operation, it will be appreciated that the patient to be treated with the stent 15 will be prepped in the normal manner and access to the vascular system will be achieved. It will be appreciated that the delivery system catheter (not shown) may be prepared in advance by telescoping the distal end of such stent 15 over the central tubular member 31 to its mounting region 29. The stent 15 may then be compressed radially inwardly by its thermally sensitive bias, or by an external compression apparatus (not shown) around the stent mounting region 29. As shown in FIG. 6, to configure the system, the individual stop wings 52 are rotated radially inwardly by their thermally sensitive bias or by an external force so that they rest against the stent tubular central tubular member 31 in an orientation that is substantially parallel to the longitudinal axis of the stent body 40. The sheath 34 may then be drawn proximally over the central tubular member 31 to encompass the stent 15 in its compressed state, and the stop container tube 36 may be advanced distally to encompass the undeployed stop wings 52 in their retracted position. The delivery system 28 is then prepared to be inserted into the patient's vasculature for delivery of the stent to the stenotic region 29.

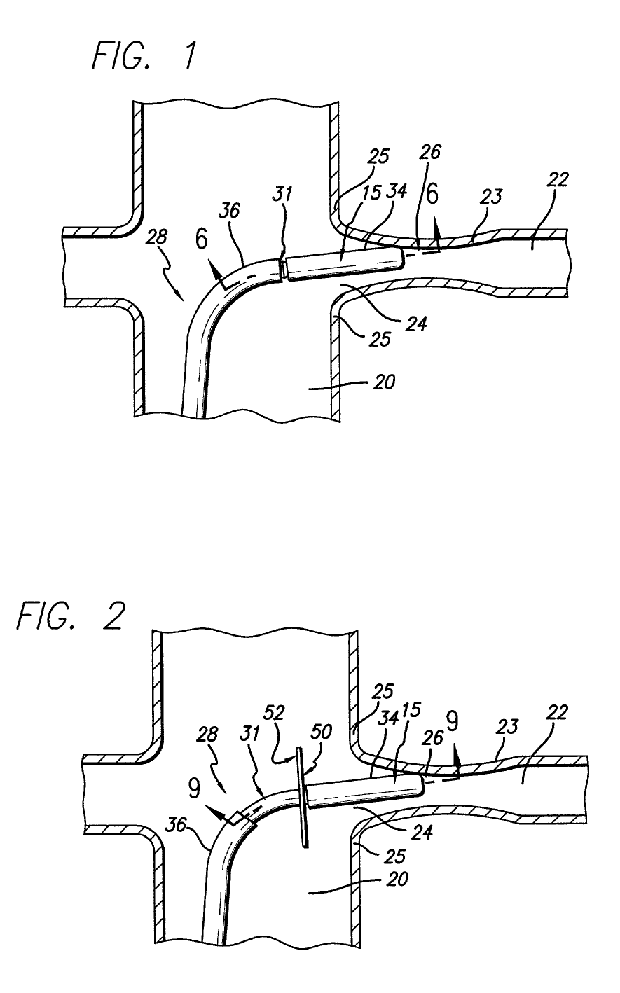

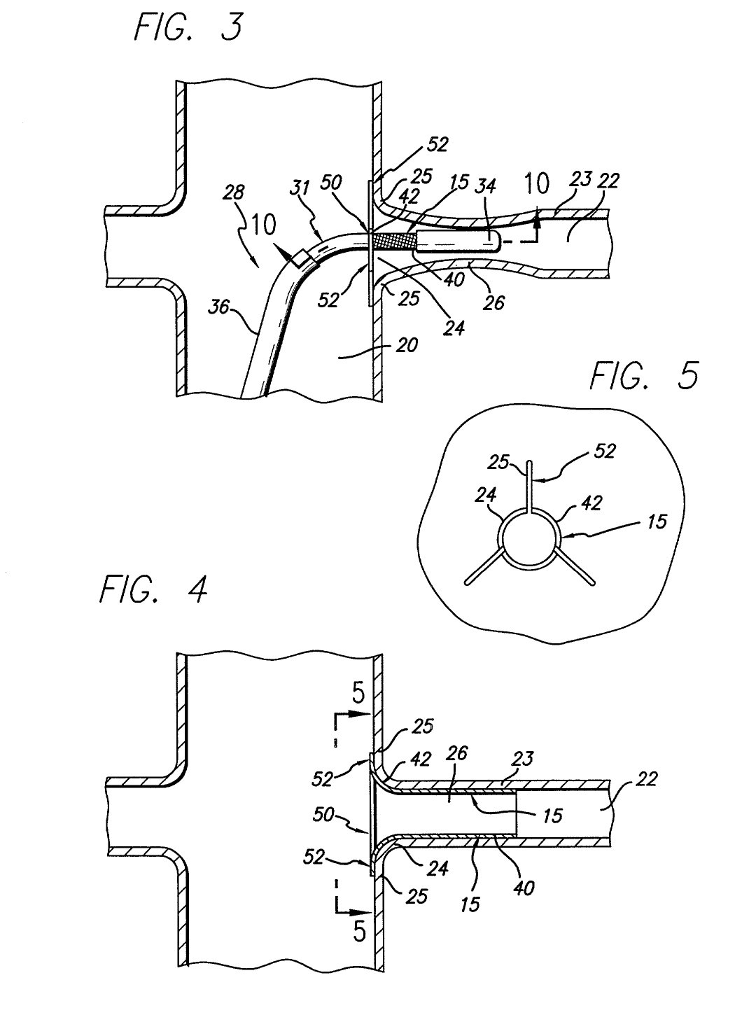

A guide wire is advanced to the stent deployment region in a conventional manner and advancing the delivery catheter (not shown) thereover to carry the stent through the aorta 20 and establish a location adjacent to the ostium 24 of the renal artery 22, as shown in FIG. 1. The doctor may then grasp the stop container tube 36 at its proximal end, and, while holding the central tubular member 31, retract the stop container tube proximally to the position shown in FIG. 2, thus unsheathing the stop wings 52. The thermally responsive hinges 42 will then be heated to approach the body temperature of the patient, thus causing the stop wings 52 to deploy radially outwardly to the position shown in FIG. 2. As shown in FIGS. 2 and 3, the delivery system 28 may then be further advanced such that, as the sheathed stent body 40 is advanced progressively into the ostium 24 and into the root of the renal artery 22, the deployed stop wings 52 engage the wall 25 of the aorta 20 surrounding the ostium, thereby defining a limit of advancement within the renal artery for the sheathed stent 15. It is contemplated that radiopaque markers may be placed in the vicinity of the stop wings and the distal extent of the stent 15 to assist in observing the progress of the advancement. The doctor should feel the resistance afforded to the delivery system 28 once the deployed stop wings engage the interior of the aorta wall 25 to positively stop the stent body 40 in the position shown in FIG. 3 at the root of the renal artery.

While holding the central tubular member 36 firmly at the proximal extremity, the retractor wire 64 is withdrawn to move the sheath 34 distally as shown in FIGS. 3 and 10. As the retractor wire is further withdrawn to the position shown in FIG. 11, the sheath is moved to its inside-out position shown also in FIG. 11, thereby fully releasing the stent body 40. The exposed stent body 40 is constructed such that it will then, under the influence of the warming temperature of the patient's body, radially expand from the distal to the proximal end into the interior wall 23 of the renal artery 22 as shown in FIG. 4. It will be appreciated that the stent will be positioned within the diseased renal artery 22 in the desired position, having gradually expanded around the central tubular member 31 such that the stent 15 is frictionally held in position within the lumen by the renal artery wall 23 and securely held in position within the ostium 24 by the stop wings 52 as they positively engage the surrounding aorta wall 25 as shown in FIG. 5. The diseased renal artery 22 is thereby repaired, and the blood flow through both renal artery and the aorta 20 is restored. The stop container tube 36, retractor wire 64, central tubular member 31, and delivery catheter are then withdrawn from the aorta and the vasculature.

While several forms of the present invention have been illustrated and described, it will also be apparent that various modifications may be made without departing from the spirit and scope of the invention. For instance, it will appreciated that various forms of sheaths may be employed for encompassing the undeployed stop wings 52 and compressed stent 15 during delivery. Additionally, it will also be appreciated that the stent and stop wings may consist of non-temperature sensitive self-expanding material that facilitates the deployment of the stop wings 52, with the assistance of the outwardly biased hinges 42, and expands the stent 15 in the absence of an external restraining device such as a sheath 34. In such an embodiment, the delivery system may comprise a central tubular member 31 that is disposed around a sheath tubular member (not shown) that has an elongated sheath 34 attached to its distal extremity. Such elongated sheath may extend proximally therefrom externally over the central tubular member 31, encompassing the compressed stent 15 and the undeployed stop wings 52 in their retracted position, thereby resembling an umbrella in its retracted state. In this embodiment, the sheath 34 is incorporated into a distal tip section (not shown) that is fixably mounted to the end of a sheath tubular member such that the sheath tubular member may be distally advanced to distally advance the sheath 34 and expose the stent 15 for expansion.

In such an embodiment, the system 28 is prepared for delivery by advancing the sheath tubular member distally to distally advance the sheath 34, exposing the stent mounting region 29 as the sheath tubular member is telescopically advanced beyond the distal extremity of the central tubular member 31. The stent 15 may then be compressed radially inwardly by an external compression apparatus (not shown) around the stent mounting region 29. To further configure the system, the individual stop wings 52 are rotated radially inwardly by an external force such that they rest against the stent tubular body 40 in an orientation that is substantially parallel to its longitudinal axis. The sheath tubular member may then be drawn proximally within the central tubular member to secure the stent in its compressed state and the stop wings in their retracted position for delivery. As the sheath moves to cover the compressed stent and the undeployed stop wings, the sheath exerts a radially inward force, compressing the stop wings radially inwardly so that they rest against the tubular body 40 of the compressed stent and holding the stent in its compressed state for delivery. It is contemplated that a the stop wings may be convex in shape to permit their complementary receipt by the stent tubular body when the stop wings are resting against the stent tubular body 40 in their undeployed position 56.

The central tubular member 31 is then ready for insertion into the proximal end of the delivery catheter (not shown) to deliver the stent 15 to the stenotic site 26 within the vasculature. For renal angioplasty, the delivery catheter may be advanced through the aorta 20 until it is positioned adjacent to the ostium 24 at the intersection of the aorta and the diseased renal artery 22. Once the stent has been positioned within the branch vessel wall relative to the stenotic site, the doctor may grasp the proximal end of the delivery system catheter 30 and the sheath tubular member 35 and gradually advance the sheath tubular member distally. As the sheath tubular member is advanced, the sheath 34 is also distally advanced to uncover the undeployed stop wings 52. The outwardly biased hinges 48 then rotate the uncovered stop wings radially outwardly to their deployed transverse position 56. The central tubular member is then advanced into the diseased branch vessel 22, with the stop wings 52 deployed and the sheath 34 holding the stent body compressed. This advancement culminates when the deployed stop wings 52 engage the aorta wall 21 that surrounds the ostium 24, thereby securing the stent in the renal artery 22 and positioning it for expansion.

It will be appreciated that any of the stop wings directed longitudinally relative to the aorta may retain their normal deployed position projecting perpendicular to the axis of the stent body 40. The stop wings 52 projecting lateral to the axis of the aorta 20 may be flexed proximally to angle proximally down from the base thereof. With the stop wings abutted against the wall of the aorta to register the stent body 40 of such stent 15 with the diseased wall of the renal artery 22 adjacent the ostium 24, the sheath tubular member 35 may be advanced to further advance the sheath 34 distally and expose the stent body 40, thereby permitting its expansion within the diseased vessel. Whether under influence of raising temperatures or its own inherent memory, the stent body will gradually grow in a radial outward direction. Such stent may be structured to progressively expand from its proximal end to its distal end.

The sheath tubular member is withdrawn telescopically into the central tubular member 31, and the central tubular member and sheath tubular member are telescopically withdrawn in unison back through the inner diameter of the expanded stent 15, and the delivery system 28 is withdrawn from the vasculature.

In another embodiment, it is also contemplated that the sheath 34 only encompass the stop wings 52, and it is withdrawn towards the proximal end of the delivery catheter 30 to expose the stop wings 52 so that they pivot to their deployed position 56. In this embodiment, the stent may be expanded by a balloon 32 mounted on the delivery system catheter 30, or may expand on its own by virtue of its self-expanding material composition and the outwardly biased hinges 42. In all embodiments that rely on a sheath 34 to hold the stent 15 in its compressed state for delivery, it is also contemplated that a lubricous fluid may be added between the outer diameter of the compressed stent and the inner diameter of the sheath 34 to reduce the frictional forces created during removal of the sheath 30. Further, in alternative applications, the sheath may only span the longitudinal extent of the stent and retracted stop wings 52, and may be retracted from the proximal end of the delivery system through the manipulation of a control wire that extends along the length of the delivery system through an inner lumen and is connected to the proximal or distal end of the sheath.

Additionally, it will be appreciated by those skilled in the art that the expansion of the stent 15 and the deployment of the stop wings 52 may be effected by additional embodiments of the present invention. For example, the stent 15 and stop wings 52 may be comprised of a shape-memory material such as NiTi. At temperatures below the patient's body temperature, the stent body 40 will assume its compressed position with the stop wings 52 retracted along the exterior surface thereof or along the surface of the central tubular member 31. The NiTi alloy is in its martensitic state at temperatures below the patient's body temperature where it is malleable and flexible for delivery through the vasculature. At an increased temperature, the stent 15 expands and the stop wings 52 deploy. At elevated temperatures, the NiTi alloy reverts to stable austenite so that the stent expands and the stop wings pivot open and become rigid enough to support the vessel and provide a positive stop. The doctor may maintain the cooler temperature during delivery by utilizing a sheath to act as a heat sink to delay heating and expansion thereof, and may then expose the stent 15 and stop wings 52 to increased temperature as the stent 15 is positioned adjacent to the ostium 24. In such an embodiment, the stent body may be constructed such that it is responsive to a greater quantity of heat transfer to assume the expanded state so that expansion of the body of the stent is delayed beyond that of the hinges 48 mounting the stop wings 52. The doctor may thus facilitate the radially outwardly rotation of the stop wings 52 to their deployed position 56, and then advance the stent 15 into the branch vessel 22 as it continues to expand and the stop wings 52 engage the great vessel wall 25 surrounding the ostium 24. By further exposing the stent to the increased temperature, the stent continues to expand so that the external diameter of its tubular body 40 engages the walls of the diseased branch vessel 22 as the stop wings further secure the stent in the branch vessel.

Alternatively, instead of using a shape-memory nickel-titanium material having a phase transformation induced by temperature, a superelastic or pseudoelastic shape-memory alloy can be used. For example, an NiTi alloy having pseudoelastic properties is used to form the stent 15 and the stop wings 52 in the same manner as previously described. Instead of a temperature transformation, however, stress induced martensite is formed when the stent is compressed and when the stop wings are pivoted onto the stent and restrained by the sheath 34. After the stent has been delivered by the catheter as previously described, the sheath is withdrawn proximally thereby relieving the stress and the pseudoelastic properties provide a large degree of recoverable strain so that the stent expands radially outwardly and the stop wings pivot open. In the expanded condition, the NiTi alloy converts from stress induced martensite to the more stable austenite phase so that the stent is able to support the body lumen and the stop wings provide enough resistance in their open condition to press against the ostium as previously described.

It is also contemplated that only a portion of the stent and/or stop wings be comprised of a superelastic or shape-memory material. For example, the stop wings 52 may be the only component composed of such a material and an external expansion means, such as a balloon 32, may be utilized to expand the stent 15 or the portion of the stent that is not composed of the superelastic or shape-memory material. Such materials can include stainless steel, tantalum, titanium, cobalt-chromium, and other similar biocompatible materials.

In such an alternative embodiment, where the stent is composed of a non-self-expanding material, advancement of the stent 15 to the stenotic region 26 is accomplished by a delivery system catheter in the form of a conventional balloon catheter comprising the structure and features that are typical to balloon catheters found in the art. The stent is compressed around the balloon, and the balloon catheter is capable of securely holding the stent in place during advancement and capable of expanding sufficiently to seat the stent within the diseased branch vessel 22. In general, the balloon catheter embodies a balloon and a hollow tubular member extending proximally therefrom. The tubular member is in fluid communication with the balloon providing a means for expansion and deflation of the balloon. The tubular member is also comprised of sufficient rigidity to facility advancement of the balloon catheter and the stent through the patient's vasculature. It is contemplated that the tubular member may also embody a secondary cavity through which a guide wire (not shown) may be passed.

From the foregoing, it will be appreciated that the stent of the present invention may be reliably and accurately placed in a stenotic region 26 of a or a branch vessel such as the renal artery 22 to facilitate blood flow to the renal artery 22 and the adjacent great vessel such as the aorta 24. Additionally, the deployable stop 50 comprising resilient stop wings 52 ensures that the stent is securely seated in the renal artery 22 and makes delivery and deployment of the stent 15 more accurate and reliable.

When stress is applied to a specimen of a metal such as nitinol exhibiting superelastic characteristics at a temperature at or above that which the transformation of the martensitic phase to the austenitic phase is complete, the specimen deforms elastically until it reaches a particular stress level where the alloy then undergoes a stress-induced phase transformation from the austenitic phase to the martensitic phase. As the phase transformation progresses, the alloy undergoes significant increases in strain with little or no corresponding increases in stress. The strain increases while the stress remains essentially constant until the transformation of the austenitic phase to the martensitic phase is complete. Thereafter, further increase in stress is necessary to cause further deformation. The martensitic metal first yields elastically upon the application of additional stress and then plastically with permanent residual deformation.

If the load on the specimen is removed before any permanent deformation has occurred, the martensite specimen will elastically recover and transform back to the austenitic phase. The reduction in stress first causes a decrease in strain. As stress reduction reaches the level at which the martensitic phase transforms back into the austenitic phase, the stress level in the specimen will remain essentially constant (but less than the constant stress level at which the austenitic crystalline structure transforms to the martensitic crystalline structure until the transformation back to the austenitic phase is complete); i.e., there is significant recovery in strain with only negligible corresponding stress reduction. After the transformation back to austenite is complete, further stress reduction results in elastic strain reduction. This ability to incur significant strain at relatively constant stress upon the application of a load and to recover from the deformation upon the removal of the load is commonly referred to as superelasticity.

As introduced above, an exemplary stent of the present invention includes a superelastic material. The term “superelastic” refers to an isothermal transformation, more specifically stress inducing a martensitic from an austenitic phase. Alloys having superelastic properties generally have at least two phases: a martensitic phase, which has a relatively low tensile strength and which is stable at relatively low temperatures, and an austenitic phase, which has a relatively high tensile strength and which is stable at temperatures higher than the martensitic phase. Superelastic characteristics generally allow the metal stent to be deformed by collapsing and deforming the stent and creating stress which causes the NiTi to change to the martensitic phase. The stent is restrained in the deformed condition to facilitate the insertion into a patient's body, with such deformation causing the phase transformation. Once within the body lumen, the restraint on the stent is removed, thereby reducing the stress therein so that the superelastic stent can return to its original undeformed shape by the transformation back to the austenitic phase.

Alternatively, the stent body and the stop wings can be formed of a resilient material such as stainless steel that has been heavily cold worked so that the material acts as a spring. For example, the stainless steel can be cold worked so that the stent can be compressed and the stop wings compressed against a catheter shaft and held in place by a sheath that covers the stent and the stop wings. When the external restraining device such as a sheath is withdrawn after positioning the catheter in the target vessel, the stent will self expand and the stop wings will deploy to engage the great vessel wall surrounding the ostium.

While a particular form of the invention has been illustrated and described, it will also be apparent to those skilled in the art that various modifications and changes can be made with regard to the foregoing detailed descriptions without departing from the spirit and scope of the invention. Accordingly, it is not intended that the invention be limited except by the appended claims.

What is claimed:

1. An expandable stent to be advanced intralumenally through a great vessel to the ostium of a branch vessel and implanted therein, comprising:
an elongated tubular body having a proximal end and a distal end, the tubular body having a diameter sized for delivery through the ostium to be positioned in the branch artery and being expandable from a compressed position to an implanted position; and
an elongated stent stop mounted adjacent the tubular body proximal end and configured to be deployed in the great vessel from an undeployed position coextensive with the wall of the tubular body to a transversely projecting deployed position projecting laterally from the tubular body to, upon the tubular body being advanced into the branch vessel, engage the wall of the great vessel to limit further distal advancement of the stent.

2. The expandable stent of claim 1, wherein the stent stop is constructed to be biased towards the deployed position.

3. The expandable stent of claim 1, wherein the stent stop aligns with a longitudinal axis of the tubular body when in the compressed position.

4. The expandable stent of claim 1, wherein the tubular body is constructed of a shape-memory material.

5. The expandable stent of claim 4, wherein the tubular body is constructed of temperature responsive shape-memory material to retain the tubular body in the contracted position.

6. The expandable stent of claim 4, wherein the shape-memory material expands in response to body temperature to expand to the implanted position.

7. The expandable stent of claim 1, wherein the stent is formed from a superelastic material.

8. The expandable stent of claim 7, wherein the superelastic material expands isothermally in response to the release of stress on the tubular body.

9. The expandable stent of claim 1, wherein the stent is formed from a spring-like material.

10. The expandable stent of claim 9, wherein the spring-like material is configured to self-expand the stent to the implanted state.

11. The expandable stent of claim 1, wherein the stent stop includes a plurality of resilient stop wings.

12. The expandable stent of claim 1, wherein the stent stop includes three resilient stop wings that project radially outwardly from the stent body at an angle of substantially 90° relative to a longitudinal axis of the tubular body.

13. The expandable stent of claim 12, wherein each of the stop wings having an interior surface positioned adjacent an exterior surface of the stent body.

14. The expandable stent of claim 1, wherein the stop is mounted on the proximal end of the stent body.

15. The stent of claim 1, wherein the stent stop includes a plurality of resilient stop wings projecting radially outwardly from the longitudinal axis of the stent body and defining a combined diametrical length greater than the diameter of the ostium.

16. A stent and catheter delivery system for repairing a branch vessel branching off from a great vessel, comprising:
a stent including an elongated tubular body having a compressed position and being expandable to an implanted position for engaging the walls of the branch vessel, the stent further including a stop at a proximal end of the tubular body pivotable from a retracted position to a deployed position projecting laterally relative to the longitudinal axis of the tubular body to engage the wall of the great vessel on the opposite sides of the ostium;
a catheter having a mounting region at the distal extremity for mounting the stent thereon;
a retainer sheath to cover and retain the stent on the mounting region, and
a retractor member associated with the retainer sheath and being moveable to withdraw the retainer off of the stent to release the stent for expansion to the implanted position.

17. The system of claim 16, wherein such stent body is constructed of memory retaining material normally assuming such expanded position.

18. The system of claim 16, wherein the retractor member withdraws the sheath distally to uncover and release the stent.

19. The system of claim 16, wherein a retainer tube telescopes over such catheter from the proximal end to engage and retain the stops in the retracted position.

20. A method for delivery of an expandable stent to a branch vessel branching outwardly from an ostium of a great vessel, comprising:
providing a stent having an elongated tubular body which is expandable to an implanted position in a vessel and further having an elongated stop adjacent a proximal end thereof;
mounting the stent on a distal end of a catheter;
introducing and advancing the catheter into the great artery;
deploying the stop from a retracted position to a deployed position projecting laterally relative to the longitudinal axis of the stent to engage the wall of the great vessel at the ostium;
further advancing the catheter to advance the stent into the branch vessel a distance sufficient to engage the stop with the wall of the great vessel; and
expanding the stent into contact with the wall of the branch vessel.

21. The method of claim 20, wherein the stop projects substantially perpendicular relative to the longitudinal axis of the stent.

22. The method of claim 20, wherein mounting the stent includes fitting a sheath over the stent; and
after deploying the stop and engaging the wall of the great vessel, removing the sheath from the stent.

23. The method of claim 20, wherein mounting the stent further includes advancing a stop retainer over the stop to restrain the stop in the retracted position.
